(19) United States
(12) Patent Application Publication
(10) Pub. No.: US 2026/0177481 A1
(43) Pub. Date: Jun. 25, 2026

(54) DEVICE FOR SENSING WATER QUALITY

(71) Applicant: LG ELECTRONICS INC., Seoul (KR)

(72) Inventor: Jeonghoon CHO, Seoul (KR)

(73) Assignee: LG ELECTRONICS INC., Seoul (KR)

(21) Appl. No.: 19/261,218

(22) Filed: Jul. 7, 2025

(30) Foreign Application Priority Data
Dec. 19, 2024 (WO) PCT/KR2024/020744

(51) Int. Cl. G01N 21/51; C02F 1/00; G01N 21/59; G01N 33/18

(52) U.S. Cl. CPC G01N 21/51; C02F 1/008; G01N 21/59; G01N 33/18; C02F 2209/003; C02F 2209/02; C02F 2209/11; C02F 2303/14; G01N 2201/061; G01N 2201/0636; G01N 2201/123

(57) ABSTRACT

Proposed is device for sensing water quality, the device including a fluid channel pipe, an external light blocking structure formed such that the fluid channel pipe is inserted into the external light blocking structure, and configured such that an inlet and an outlet of the fluid channel pipe are exposed at first and second lateral surfaces, respectively, a first light source arranged at a first point on the fluid channel pipe and configured to emit first light into an internal region of the fluid channel pipe, a second light source arranged at a second point on the fluid channel pipe and configured to emit second light into the internal region, and a detector configured to detect scattered light resulting from the first light being scattered within the internal region, and to detect reflected light resulting from the second light being reflected by a surface of the fluid channel pipe.

### CROSS-REFERENCE TO RELATED APPLICATION

Pursuant to 35 U.S.C. § 119(a), this application claims the benefit of the earlier filing date and the right of priority to PCT Patent Application No. PCT/KR2024/020744, filed on Dec. 19, 2024, the contents of which are incorporated by reference herein in their entirety.

### TECHNICAL FIELD

The present disclosure relates to a device for sensing water quality. More specifically, the present disclosure relates to a device for sensing water quality, the device being used in a household appliance and being capable of measuring the turbidity of water, and a method of sensing water quality.

### BACKGROUND

In general, household appliances that use water, such as water purifiers, dishwashers, and washing machines, are required to use clean water. Therefore, various sensors for monitoring the turbidity of water are mounted within these appliances.

Turbidity refers to the concentration of light-scattering particles or light-absorbing particles suspended in a fluid. In a case where the turbidity of the fluid increases, the light transmissivity may vary depending on factors such as the distribution of suspended particles within the fluid, the refractive index of the particles, and the surface characteristics of the particles.

Waste of resources such as water, electricity, and detergent can be minimized by adjusting the cleaning or filtration cycles of household appliances based on water turbidity information. In addition, drinking water purified under optimal conditions using water turbidity information can be provided, or articles, such as dishware and clothing, that are cleaned under optimal conditions can be provided.

However, existing turbidity sensors have limitations in measuring water pollution in a low-turbidity region, thereby rendering it difficult to ensure the safety of potable water as required for household drinking water appliances. Therefore, it is necessary to develop a device for measuring water quality, which is capable of performing a sensing function that detects not only the quality of water in the high-turbidity region, but also the quality of water in a low-turbidity region.

A system for detecting impurities using chaotic waves can be provided to measure water quality. In this regard, when the depth of the concavo-convex profile is small relative to the wavelength of incident light, the frequency of multiple scattering events caused by reflection decreases. Conversely, when the depth of the concavo-convex profile is large relative to the wavelength of incident light, the frequency of multiple scattering events caused by reflection increases. Therefore, interference with an optical path caused by movements of constituent materials within the fluid, and changes in patterns over time can be measured by emitting light, subject to multiple scattering, into a fluid. Accordingly, it is possible to estimate the presence or absence of impurities within the fluid and the concentration of the impurities.

However, when a regular pattern or an irregular concavo-convex structure is employed to detect low turbidity in the low-turbidity region through the use of multiple reflections of light, there is an increased likelihood that the flow of water will be obstructed and that foreign materials will accumulate on a portion of the concavo-convex profile. In this regard, patterns resulting from interference of laser light induced by impurities can be analyzed. Therefore, although it is possible to estimate the concentration of impurities or foreign materials within a fluid or the turbidity of the fluid, this estimation cannot be performed in real time due to a time delay phenomenon.

### SUMMARY

One object of the present disclosure is to provide a device for measuring water quality, the device being capable of performing a sensing function that detects the quality of low-turbidity water.

Another object of the present disclosure is to enable real-time turbidity measurement by preventing a time delay phenomenon when estimating the turbidity of a fluid due to foreign materials within the fluid.

A further object of the present disclosure is to provide a device for measuring water quality, the device being capable of effectively detecting foreign materials with scarring and reflecting components.

Another object of the present disclosure is to effectively determine an abnormal state of water quality, a contaminated state, or other irregular water quality conditions in association with the measurement of the turbidity of water.

Still another object of the present disclosure is to overcome erroneous detection issues, caused by water quality conditions such as biofilms and activated carbon in existing sensors in a low-turbidity region applicable to household drinking water appliances.

Yet another object of the present disclosure is to provide a household appliance such as an apparatus for purifying water, the apparatus being capable of detecting the quality of low-turbidity water and measuring the turbidity of water in real time.

According to one aspect of the present disclosure, there is provided a device for sensing water quality, the device including: a fluid channel pipe; an external light blocking structure formed such that the external light blocking structure surrounds the fluid channel pipe and that the fluid channel pipe is inserted into the external light blocking structure, and configured such that an inlet and an outlet of the fluid channel pipe are exposed at a first lateral surface and a second lateral surface, respectively; a first light source arranged at a first point on the fluid channel pipe and configured to emit first light into an internal region of the fluid channel pipe; a second light source arranged at a second point on the fluid channel pipe and configured to emit second light into the internal region of the fluid channel pipe; and a detector configured to detect scattered light resulting from the first light being scattered within the internal region of the fluid channel pipe, and to detect reflected light resulting from the second light being reflected by a surface of the fluid channel pipe. The device for sensing water quality may include a processor operatively coupled to the first light source, the second light source, and the detector, and configured to measure the turbidity for a liquid within the fluid channel pipe based on the detected scattered light resulting from the first light and the detected reflected light resulting from the second light and to detect whether the fluid channel pipe is contaminated.

According to an embodiment, in the device, the first point at which the first light source is arranged and a point at which the detector is arranged may be spaced apart with an angular extent of 90 degrees. The second point at which the second light source is arranged and the point at which the detector is arranged may be positioned at an arc angle of 0 degrees.

According to an embodiment, the device may further include a reflector formed along the inside or outside of the fluid channel pipe between a third point and a fourth point on the inside or outside thereof. The third point on the fluid channel pipe may be a point that is opposite the first point on the fluid channel pipe, and the fourth point on the fluid channel pipe may be a point that is opposite the second point on the fluid channel pipe. The reflector may be formed in a first sector shape spanning an angular extent of 90 degrees or less along a curved surface of the fluid channel pipe between the third point and the fourth point on the fluid channel pipe.

According to an embodiment, the device may further include a reflector formed in opposite directions from the fourth point along the inside or outside of the fluid channel pipe. The fourth point on the fluid channel pipe may be a point that is opposite the second point on the fluid channel pipe. The reflector may be formed in a second sector shape spanning an angular extent of 90 degrees or less along a curved surface of the fluid channel pipe, the reflector being centered at the fourth point.

According to an embodiment, the device may further include a reflector formed in a third sector shape spanning an angular extent from more than 90 degrees to less than 120 degrees along a curved surface of the fluid channel pipe between the third point and the first point on the inside or outside of the fluid channel pipe. The reflector may be formed as an asymmetrical structure that extends in opposite directions from the fourth point along the inside or outside of the fluid channel pipe. The reflector may be formed such that a first end thereof is adjacent to the third point and that a second thereof is spaced from the first point, with an angular extent of 45 degrees or more.

According to an embodiment, the device may further include a switch configured to control a first light emission state of the first light source and a second light emission state of the second light source. The processor may control the switch such that the first light source remains turned on to emit the first light into the internal region and may measure a first turbidity value by detecting the scattered light resulting from the first light. The processor may control the switch such that the second light source remains turned on to emit the second light into the internal region and may measure a second turbidity value by detecting the reflected light resulting from the second light. The processor may compute a turbidity value for the internal region based on the first turbidity value and the second turbidity value.

According to an embodiment, in the device, the processor may control the second light source to remain turned off during a first time interval during which the first light source remains turned on. The processor may measure the first turbidity value during a second time interval, subsequent to the first time interval. The processor may control the first light source to remain turned on during a third time interval during which the second light source remains turned on. The processor may measure the second turbidity value during a fourth time interval, subsequent to the third time interval.

According to an embodiment, the device may further include a digital-to-analog converter that is arranged between the processor and the switch, receives a serial data signal and a serial clock signal from the processor, and transfers control signals for controlling the first light source and the second light source, through the switch. The device may further include an operational amplifier connected to the detector and configured to amplify signals associated with the detected scattered light and the detected reflected light. The device may further include a low-pass filter configured to filter the amplified signals such that low-frequency signal components are passed through and that the passed low-frequency signal components are transferred to the processor.

According to an embodiment, the device may further include a thermistor coupled to the processor and configured to measure the temperature of the internal region of the fluid channel pipe. The processor may determine whether the first turbidity value or the second turbidity value exceeds a threshold value. The processor may determine whether the fluid channel pipe is contaminated with condensation, when the first turbidity value or the second turbidity value exceeds the threshold value. The processor may control a temperature adjustment device such that the temperature of the internal region of the fluid channel pipe is controlled, when it is determined that the fluid channel pipe is contaminated with the condensation.

According to an embodiment, in the device, when the first turbidity value and the second turbidity value are less than the threshold value, the processor may determine whether the first turbidity value and the second turbidity value remain in a normal state where a linear relationship defined in the form of a linear function is established, or in an abnormal state that deviates from the linear relationship. When the first turbidity value and the second turbidity value remain normal, the processor may compute the turbidity value for the internal region based on the first turbidity value and the second turbidity value.

According to an embodiment, in the device, when the first turbidity value and the second turbidity value remain abnormal, the processor may determine whether the fluid channel pipe is contaminated with foreign materials in the internal region thereof or with biofilms. The processor may perform control such that a fluid channel of the fluid channel pipe is cleaned, when it is determined that the fluid channel pipe is in a contaminated state caused by the foreign materials in the internal region thereof or by the biofilms. The processor may perform control such that water is introduced into the internal region of the fluid channel pipe, in a state where the fluid channel of the fluid channel pipe is cleaned.

According to an embodiment, in the device, the threshold value for the first turbidity value or the second turbidity value may be set to 4,000. The normal state may be defined as a first region where the first turbidity value and the second turbidity value form the linear relationship defined in the form of a linear function. The normal state may be defined as a second region where the first turbidity value is greater than in the first region, or as a third region where the second turbidity value is greater than in the first region. The contaminated state may be defined as a fourth region where the first turbidity value and the second turbidity value are greater than those associated with the abnormal state of the third region.

According to an embodiment, in the device, in a state where the temperature of the internal region is controlled, the processor may control the switch such that the first light source remains turned on to emit the first light into the internal region. The processor may measure the first turbidity value by detecting the scattered light resulting from the first light. The processor may control the switch such that the second light source remains turned on to emit the second light into the internal region. The processor may measure the second turbidity value by detecting the reflected light resulting from the second light. The processor may compute the turbidity value for the internal region based on the first turbidity value and the second turbidity value, when the measured first turbidity value or the measured second turbidity value remains normal.

According to an embodiment, in the device, when the first turbidity value and the second turbidity value are less than the threshold value, the processor may determine whether the fluid channel pipe is contaminated with foreign materials in the internal region or with biofilms. The processor may perform control such that a fluid channel of the fluid channel pipe is cleaned, when it is determined that the fluid channel pipe is contaminated with the foreign materials in the internal region thereof or with the biofilms. The processor may perform control such that water is introduced into the internal region of the fluid channel pipe, in a state where the fluid channel of the fluid channel pipe is cleaned.

According to an embodiment, in the device, the processor may control the switch such that the first light source remains turned on to emit the first light into the internal region in a state where water is introduced into the internal region. The processor may measure the first turbidity value by detecting the scattered light resulting from the first light. The processor may control the switch such that the second light source remains turned on to emit the second light into the internal region. The processor may measure the second turbidity value by detecting the reflected light resulting from the second light. The processor may compute the turbidity value for the internal region based on the first turbidity value and the second turbidity value, when the measured first turbidity value or the measured second turbidity value remains normal.

According to another aspect of the present disclosure, there is provided a method of sensing water quality, the method being performed by a processor of a device for sensing water quality. The method includes: controlling a switch such that a first light source arranged at a first point on a fluid channel pipe remains turned on to emit first light into an internal region of the fluid channel pipe; measuring a first turbidity value by enabling a detector to detect scattered light resulting from the first light being scattered within the internal region; controlling the switch such that a second light source arranged at a second point on the fluid channel pipe remains turned on to emit second light into the internal region; measuring a second turbidity value by enabling the detector to detect reflected light resulting from the second light being reflected by a surface of the fluid channel pipe; and computing a turbidity value for the internal region based on the first turbidity value and the second turbidity value.

According to a further aspect of the present disclosure, there is provided an apparatus for purifying water, the apparatus including: a water intake port formed such that water is introduced therethrough; a device for sensing water quality, the device configured to detect the turbidity of the water flowing through a fluid channel pipe from the water intake port; and a water supply port formed such that water, the turbidity of which is detected, is discharged therethrough. The device for sensing water quality includes: a fluid channel pipe; an external light blocking structure formed such that the external light blocking structure surrounds the fluid channel pipe and that the fluid channel pipe is inserted into the external light blocking structure, and configured such that an inlet and an outlet of the fluid channel pipe are exposed at a first lateral surface and a second lateral surface, respectively; a first light source arranged at a first point on the fluid channel pipe and configured to emit first light into an internal region of the fluid channel pipe; a second light source arranged at a second point on the fluid channel pipe and configured to emit second light into the internal region of the fluid channel pipe; a detector configured to detect scattered light resulting from the first light being scattered within the internal region of the fluid channel pipe, and to detect reflected light resulting from the second light being reflected by a surface of the fluid channel pipe; and a processor operatively coupled to the first light source, the second light source, and the detector, and configured to measure the turbidity of a liquid within the fluid channel pipe. The processor may measure a first turbidity value by enabling a detector to detect the scattered light resulting from the first light, measure a second turbidity value by enabling the detector to detect the reflected light resulting from the second light, compute a turbidity value for the internal region based on the first turbidity value and the second turbidity value, and detect whether the fluid channel pipe is contaminated.

According to the present disclosure, there is provided a device for measuring water quality, the device being capable of performing a sensing function that detects the quality of low-turbidity water using scattered light and reflected light that result from the scattering and reflection of incident light by foreign materials.

According to the present disclosure, water turbidity can be measured in real time by preventing a time delay phenomenon when estimating the water turbidity caused by foreign materials in a fluid, using the scattered light and the reflected light that result from the scattering and reflection of the incident light by the foreign materials.

According to the present disclosure, foreign materials having scattering and reflecting components can be effectively detected by optimally arranging two light sources and one detector in one region of a fluid channel pipe and optimally arranging a reflector on the other region thereof.

According to the present disclosure, an abnormal state of water quality and a contaminated state of water can be effectively determined through a two turbidity pattern resulting from the scattered light and the reflected light.

According to the present disclosure, erroneous detection issues, caused by water quality conditions such as biofilms and activated carbon in existing sensors, can be overcome in a low-turbidity region applicable to household drinking water appliances by identifying a machine-learning-based two-dimensional turbidity pattern.

According to the present disclosure, the turbidity of water in household drinking water appliances can be detected, thereby contributing to the safety of potable water. The safety of potable water can be ensured by preventing erroneous detection caused by contamination of a fluid channel and detecting the turbidity of water.

According to the present disclosure, a customized filter replacement cycle can be set based on contamination standards by preventing erroneous detection caused by the contamination of the fluid channel and detecting the turbidity of water. As a result, unnecessary replacement costs can be reduced.

According to the present disclosure, a cost-effective device for detecting water quality can be manufactured using low-cost light-emitting diodes and photodiodes, thereby expanding its applications in household appliances.

According to the present disclosure, there is provided a household appliance, such as an apparatus for purifying water, which is capable of detecting low-turbidity water and measuring the turbidity of water in real time by employing an optimal structure including two light sources and one detector.

According to the present disclosure, a household appliance, such as an apparatus for purifying water, can provide a service for maintaining customer satisfaction by monitoring and measuring water quality in real time, thereby controlling the water quality accordingly.

The range of possible additional applications of the present disclosure will be apparent from the detailed description provided above. However, various alterations and modifications to the present disclosure would be readily understood by a person of ordinary skill in the art without departing from the spirit and scope of the technical idea of the present disclosure. The detailed descriptions of specific embodiments, such as preferred embodiments of the disclosure, should be understood as illustrative examples only.

### BRIEF DESCRIPTION OF THE DRAWINGS

The above and other aspects, features, and additional advantages of the present disclosure will be more clearly understood from the following detailed description, which is taken in conjunction with the accompanying drawings, in which:

FIG. 1 is a view illustrating the structure of a device for sensing water quality according to the present disclosure having a fluid channel pipe inserted therein;

FIG. 2 is a view illustrating a block diagram of the device for sensing water quality in FIG. 1;

FIG. 3 is a view illustrating the structure of the fluid channel pipe in FIG. 25 2, which enables detection of light reflected and scattered by foreign materials as a result of the first light and second light entering the fluid channel pipe;
(a) and (b) of FIG. 4 are views illustrating the structures, respectively, of the fluid channel pipe of the device for sensing water quality, in which reflectors according to embodiments are formed in different regions, respectively, of a curved surface of the fluid channel pipe;

FIG. 5 is a graph showing the results of evaluating turbidity performance based on first scattered light and second reflected light;

FIG. 6 is a view illustrating the structure of the fluid channel pipe in which a reflector is formed in the shape of a sector spanning an angular extent of more than 90 degrees along regions of the fluid channel pipe that correspond to first and second quadrants of the XY plane;

FIG. 7 is a detailed block diagram illustrating the device for sensing water quality in FIG. 2;

FIG. 8 is a conceptual diagram for controlling the emission of light from first and second light sources and for detecting the first scattered light and the second reflected light by employing a time division scheme;

FIG. 9 is a graph that is referenced to determine a water quality state by distinguishing between two-dimensional turbidity patterns derived from a first turbidity value and a second turbidity value;

FIGS. 10 and 11 are flowcharts illustrating a method of sensing water quality according to another aspect of the present disclosure; and

FIG. 12 is a view illustrating the configuration of an apparatus for purifying water according to still another aspect of the present disclosure, which includes the device for sensing water quality.

### DETAILED DESCRIPTION OF THE EMBODIMENTS

The technology disclosed in the present specification finds application in apparatuses for purifying water and washing machines capable of suppressing scale formation. However, the technology disclosed in the present specification is not limited thereto and may also find application in all apparatuses for purifying water and washing machines capable of suppressing scale formation, insofar as the technical concept of the disclosed technology is applicable.

Technical terms used in the present specification are intended solely to describe specific embodiments and are not intended to limit the present disclosure. In addition, unless otherwise specifically defined, the technical terms used in the present specification should be construed as having the meanings generally understood by those of ordinary skill in the art to which the present disclosure pertains. These terms should not be construed either too broadly or too narrowly. In addition, when the technical term used in the present specification does not precisely reflect the technical concept of the present disclosure, it should be construed to have the meaning generally accepted by those of ordinary skill in the art. In addition, general terms used in the present specification should be interpreted based on their standard dictionary meanings or as understood within the context of this disclosure, without applying an unduly narrow construction.

In addition, terms expressed in the singular form in the present specification should be construed to include the plural form unless the context clearly dictates otherwise. Throughout the present specification, the terms such as ‘is configured to include’ or ‘includes’ should not be construed as requiring all constituent elements or all steps described herein. Rather, these terms should be construed to allow for the omission of one or more elements or steps, as well as the inclusion of one or more additional elements or steps.

Unless expressly stated otherwise, the terms ‘module’ and ‘structure’ are used interchangeably in the present specification for the purpose of description, and are not intended to denote different structures or functions.

In addition, expressions of ordinal numbers, including ‘first,’ ‘second,’ and similar, are employed for the sake of identification and convenience in description only, and are not to be construed as limiting the elements to any specific order or hierarchy. The above-mentioned terms are used solely to distinguish one element from another. For example, a first constituent element may be referred to as a second constituent element without departing from the scope of claims. Similarly, the second constituent element may be referred to as the first constituent element.

Preferred embodiments according to the present disclosure are described below with reference to the accompanying drawings. Like constituent elements are designated by like reference numerals throughout the drawings, and redundant descriptions thereof are omitted.

In addition, a detailed description of known technologies associated with the present disclosure may be omitted where such description is deemed likely to obscure or detract from the clarity of the present disclosure. In addition, the accompanying drawings are provided solely to facilitate understanding of the technical idea disclosed in the present specification. It should be noted that the accompanying drawings are not construed as imposing any limitation on the underlying technical idea.

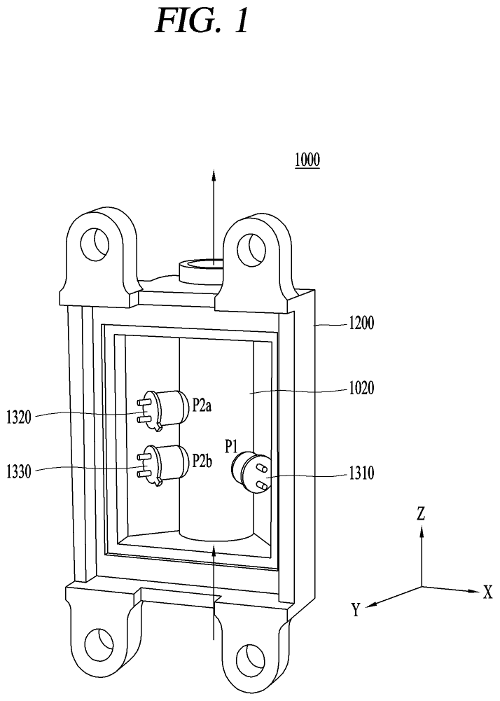

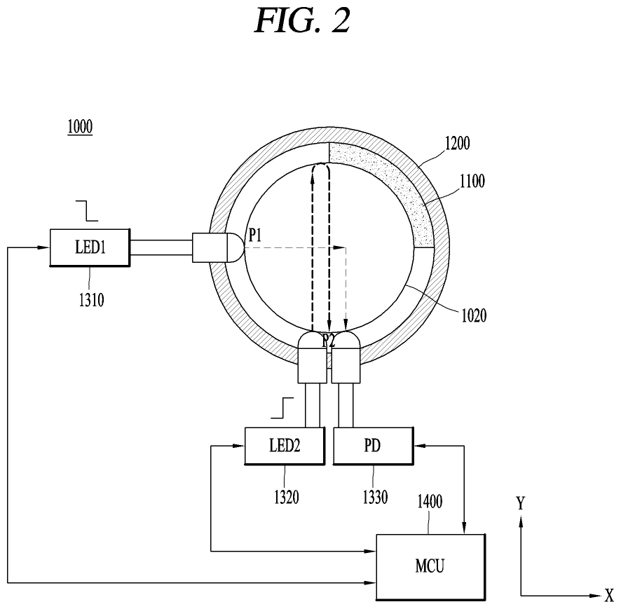

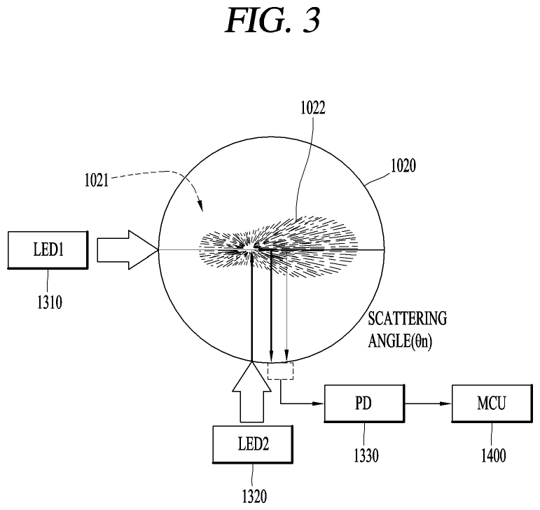

An apparatus for purifying water, which is capable of suppressing scale formation according to the present disclosure, is described below. In this regard, FIG. 1 is a view illustrating the structure of a device for sensing water quality according to the present disclosure having a fluid channel pipe inserted therein. FIG. 2 is a view illustrating a block diagram of the device for sensing water quality in FIG. 1.

With reference to a device 100 for sensing water quality in FIG. 1, a fluid channel pipe 1030 may be formed within an external light blocking structure 1200. A first light source 1310 may be arranged at a first point P1 on the fluid channel pipe 1030. A second light source 1320 may be arranged at a second point P2a on the fluid channel pipe 1030. A detector 1330 may be positioned at a second location P2b on the fluid channel pipe 1030. The second point P2a on the fluid channel pipe 1030, at which the second light source 1320 is positioned, may be higher along the Z-axis than the second point P2b on the fluid channel pipe 1030, at which the detector 1330 is positioned. In this regard, interference between the first light source 1310 and the second light source 1320 may be reduced not only on the XY plane but also along the Z-axis direction.

With reference to FIGS. 1 and 2, the device 1000 for sensing water quality according to the present disclosure is described. The device 1000 for sensing water quality is configured such that incident light emitted from a plurality of light sources is directed into the fluid channel pipe 1020. The device 1000 for sensing water quality may determine the quality of water within the fluid channel pipe 1020 by detecting light reflected and scattered by particles within the fluid channel pipe 1020 due to the incident light.

In this regard, the device 1000 for sensing water quality may be configured to include the fluid channel pipe 1020, the external light blocking structure 1200, the first light source 1310, the second light source 1320, the detector 1330, and a processor 1400. The first light source 1310 and second light source 1320 may be configured as a first light emitting diode LED and a second light emitting diode, respectively.

The external light blocking structure 1200 may be formed such that the fluid channel pipe 1020 is inserted into the external light blocking structure 1200. A liquid such as water may be accommodated within the fluid channel pipe 1020. The external light blocking structure 1200 may be formed to enclose the fluid channel pipe 1020. The external light blocking structure 1200 is configured to block the entry of light from the outside, except for light emitted from the first light source 1310 and the second light source 1320. The external light blocking structure 1200 may be configured such that an inlet and an outlet of the fluid channel pipe 1020 are exposed at a first lateral surface and a second lateral surface, respectively.

The first light source 1310 may be arranged at the first point P1 on the fluid channel pipe 1020. The first light source 1310 may be configured to emit first light into an internal region of the fluid channel pipe 1020. The second light source 1320 may be arranged at a second point P2 on the fluid channel pipe 1020. The second light source 1320 may be configured to emit the second light into the internal region of the fluid channel pipe 1020. The fluid channel pipe 1020 may be formed to have a cylindrical shape to allow fluid to be accommodated therein. The fluid channel pipe 1020 may have a tubular shape with a through-hole formed therein to allow fluid to flow therethrough, but is not limited thereto and may have other shapes depending on the intended application. The entire surface of the fluid channel pipe 1020 may be formed of a light-transmitting material.

A reflector 1100 may be arranged on one portion of the inside or outside of the fluid channel pipe 1020. The reflector 1100 can enhance the light reflection characteristics of low turbidity suspending particles that cause low light scattering. The reflector 1100 may be formed of a metallic material in such a manner as to reflect light propagating through the fluid channel pipe 1020 from the first and second light sources 1310 and 1320.

The device 1000 for sensing water quality according to the present disclosure may be configured to detect reflected and scattered light resulting from the incident first light and second light, different from each other, and thus to stably detect foreign materials. In this regard, FIG. 3 is a view illustrating the structure of the fluid channel pipe in FIG. 2, which enables detection of light reflected and scattered by foreign materials as a result of the first light and second light entering the fluid channel pipe.

With reference to FIG. 3, a liquid 1021 having turbidity may be present within the internal region of the fluid channel pipe 1020. The fluid channel pipe 1020 may be formed of glass or a plastic material. The fluid channel pipe 1020 may be configured as a vial formed from glass or plastic materials. Foreign materials 1022 may be present within the internal region the fluid channel pipe 1020. The foreign materials 1022 may float within the liquid 1021 or precipitate on the wall surface of the fluid channel pipe 1020.

The first light may be incident at an angle of 0 degrees from the first light source 1310 into the internal region of the fluid channel pipe 1020 at the first point on the fluid channel pipe 1020. The first light can be reflected by the foreign materials 1022 or scattered by the foreign materials 1022, thereby allowing first reflected light (first scattered light) to propagate in the direction of 180 degrees. In this regard, when a component of the light that is scattered by the foreign materials 1022 are greater than a component of the light that is reflected by the foreign materials 1022, the first scattered light changes its direction of propagation by an angle of approximately −90 degrees with respect to first incident light. Therefore, a scattering angle (θn) of the first scattered light may be measured as an angle of approximately −90 degrees. The scattering angle of the first scattered light is not limited thereto and may vary depending on the types and shapes of the foreign materials 1022 and the type of liquid.

The second light may be incident at an angle of 90 degrees angle from the second light source 1320 into the internal region of the fluid channel pipe 1020 at the second point on the fluid channel pipe 1020. The second light may be reflected by the foreign materials 1022 or scattered by the foreign materials 1022, thereby allowing second reflected light (second scattered light) to propagate in the direction of 180 degrees. In this regard, when a component of the light that is reflected by the foreign materials 1022 is greater than a component of the light that is scattered by the foreign materials 1022, the second reflected light changes its direction of propagation by an angle of approximately 180 degrees with respect to the first incident light. Thus, a reflection angle of the second reflected light may be measured as an angle of approximately −90 degrees.

The first scattered light, which propagates in the direction of approximately −90 degrees with respect to the first incident light, and the second reflected light, which propagates in the direction of 180 degrees with respect to the first incident light, are directed toward the second point on the fluid channel pipe 1020. The first scattered light and the second reflected light, which are directed toward the second point on the fluid channel pipe 1020, may be detected at a detector 1330 positioned at or adjacent to the second point on the fluid channel pipe 1020.

A scattered-light-based detection technique used by a sensor for measuring low turbidity may experience detection inaccuracies caused by solution states with varying water quality, as well as stability issues resulting from environmental conditions. For example, in a case where precipitates, such as biofilms, form on the wall surface of the fluid channel pipe 1020, a non-contact optical sensor may fail to detect them or output erroneous values. Therefore, in order to prevent such detection inaccuracies, it is necessary to check whether any foreign materials have precipitated.

The device 1000 for sensing water quality according to the present disclosure determines whether an abnormality is present in a water-quality detection environment by replacing a single-channel technique with a two-channel, two-dimensional array structure to achieve an improved turbidity detection limit. That is, in the case of the biofilms, the scattered light from the first light source 1310 and the reflected light from the second light source 1320 are simultaneously checked. Therefore, erroneous values may be obtained by computing the turbidity of a liquid using differences between the scattered and reflected light values of the liquid and those of typical purified water free of foreign materials.

With reference to FIGS. 1 to 3, a detection process performed by the device 1000 for sensing water quality according to the present disclosure is described in detail. The detector 1330 may be configured to detect scattered light resulting from the first light being scattered within the internal region of the fluid channel pipe 1020. In addition, the detector 1330 may be configured to detect reflected light resulting from the second light being reflected by a surface of the fluid channel pipe 1020. The processor 1400 may be operatively coupled to the first light source 1310, the second light source 1320, and the detector 1330. The processor 1400 may be configured to measure the turbidity of a liquid within the fluid channel pipe 1020. The processor 1400 may be configured to measure the turbidity of a liquid within the fluid channel pipe 1020 based on the measured scattered light resulting from the first light and the measured reflected light resulting from the second light and to detect whether the fluid channel pipe 1020 is contaminated.

The first point at which the first light source 1310 is arranged and a point at which the detector 1330 is arranged may be positioned with an angular extent of 90 degrees. The second point at which the second light source 1320 is arranged and the point at which the detector 1330 is arranged may be positioned at an arc angle of 0 degrees. The processor 1400 may be positioned on a substrate that is coupled to a lateral surface of the external light blocking structure 1200.

To detect the turbidity of a liquid caused by foreign materials in the fluid channel pipe 1020, the first light source 1310 may emit light, and scattered light, resulting from the foreign materials scattering the light, may be detected by the detector 1330. In order to detect whether the surface of the fluid channel pipe 1020 is contaminated, the second light source 1320 may emit light, and reflected light, resulting from a contamination material on the surface reflecting the light, may be detected by the detector 1330.

The reflector 1100 may be arranged on the fluid channel pipe 1020 of the device 1000 for sensing water quality. In this regard, (a) and (b) of FIG. 4 are views illustrating the structures, respectively, of the fluid channel pipe of the device for sensing water quality, in which reflectors according to embodiments are formed in different regions, respectively, of a curved surface of the fluid channel pipe. (a) and (b) of FIG. 4 illustrate that the reflector 1100 is arranged on the inside of the fluid channel pipe 1020. However, the reflector 1100 is not limited thereto and may also be positioned on the outside of the fluid channel pipe 1020 as in FIG. 2.

With reference to FIG. 2 to (a) of FIG. 4, the reflector 1100 may be arranged in a region of the inside or outside of the fluid channel pipe 1020, the region corresponding to the first quadrant of the XY plane. The reflector 1100 may be formed along the inside or outside of the fluid channel pipe 1020. The reflector 1100 may be formed along the fluid channel pipe 1020, between a third point P3 and a fourth point P4. The reflector 1100 may be arranged along the inside or outside of the fluid channel pipe 1020, between the third point P3 and the fourth point P4 on the inside or outside of the fluid channel pipe 1020. As the reflector 1100 is arranged in the region corresponding to the first quadrant of the XY plane, light scattered in the direction of the first quadrant may also be reflected and detected by the detector 1330. Accordingly, both the light scattered in the downward direction by the foreign materials 1022 and the light scattered toward the first quadrant in the upward direction may be detected by the detector 1330.

The third point P3 on the fluid channel pipe 1020 may be a point that is opposite the first point P1 on the fluid channel pipe 1020. The third point P3 on the fluid channel pipe 1020 may be a point that is opposite the first point P1 on the fluid channel pipe 1020, with an angular extent of 180 degrees. The fourth point P4 on the fluid channel pipe 1020 may be a point that is opposite the second point P2 on the fluid channel pipe 1020. The fourth point P4 on the fluid channel pipe 1020 may be a point that is opposite the second point P2 on the fluid channel pipe 1020, with an angular separation of 180 degrees. The reflector 1100 may be formed in the shape of a sector spanning an angular extent of 90 degrees or less along a curved surface of the fluid channel pipe 1020, between the third point P3 and the fourth point P4 on the fluid channel pipe 1020.

With reference to FIGS. 2 and 3 and (b) of FIG. 4, a reflector 1100b may be arranged on a region of the inside or outside of the fluid channel pipe 1020, the region corresponding to one portion of the first quadrant of the XY plane, and a region of the inside or outside of the fluid channel pipe 1020, the region corresponding to one portion of the second quadrant. The reflector 1100b may be formed along the inside or outside of the fluid channel pipe 1020. The reflector 1100b may be formed in opposite directions from the fourth point P4 along the inside or outside of the fluid channel pipe 1020.

The reflector 1100b may be formed to subtend an arc angle of 45 degrees or less along the fluid channel pipe 1020 such that the reflector 1100b is centered at the fourth point P4 and is opposite the first point P1. The reflector 1100b may be formed to subtend an arc angle of 45 degrees or less along the fluid channel pipe 1020, the reflector 1100b being centered at the fourth point P4 and facing the third point P3. The reflector 1100b may be formed to subtend an arc angle of 90 degrees or less along the inside or outside of the fluid channel pipe 1020, the reflector 1100b being centered at the fourth point P4. The reflector 1100b may be formed in the shape of a sector spanning an angular extent of 90 degrees or less along the fluid channel pipe 1020, the reflector 1100b being centered at the fourth point P4. The reflector 1100b may be formed in the shape of a sector spanning an angular extent of 90 degrees or less along the inside or outside of the fluid channel pipe 1020, the reflector 1100b being centered at the fourth point P4.

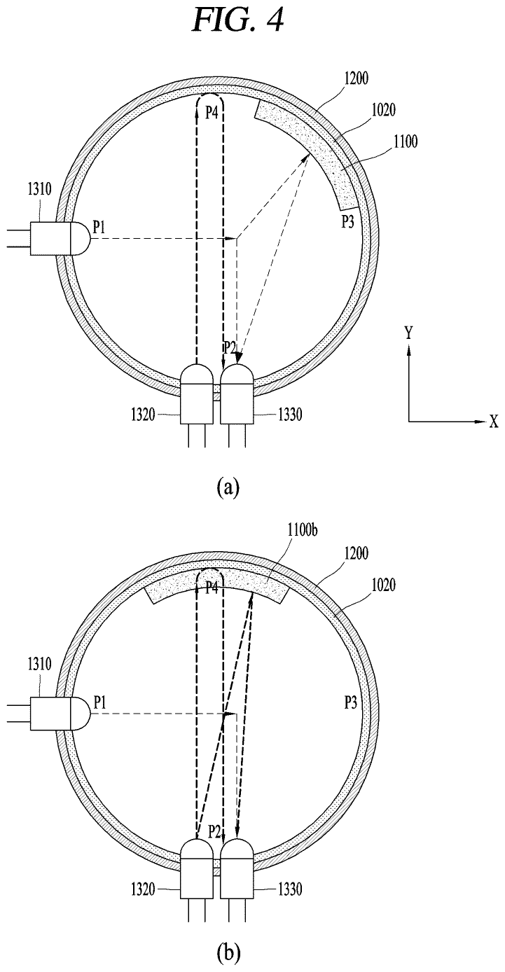

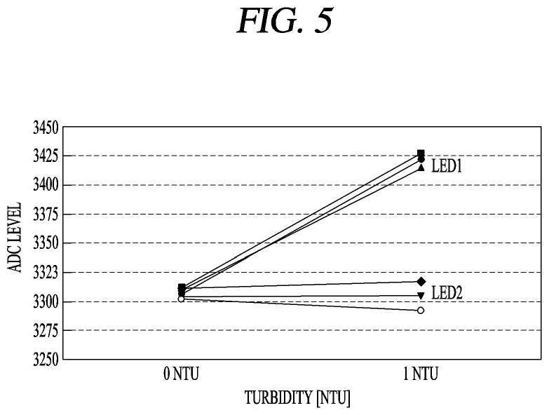

The device for sensing water quality according to the present disclosure evaluates turbidity performance based on a first turbidity value associated with the first scattered light and a second turbidity value associated with the second reflected light. In this regard, FIG. 5 is a graph showing the results of evaluating the turbidity performance based on the first scattered light and the second reflected light. With reference to FIG. 5, the X-axis and Y-axis represent turbidity and ADC level, respectively. The results of measurements based on the first scattered light from LED 1, which serves as a first light source, and the results of measurements based on the second reflected light from LED 2, which serves as a second light source are plotted together on the graph along the X-axis and the Y-axis. In this regard, the results of measurements based on the first scattered light from LED 1, which serves as the first light source, and the results of measurements based on the second reflected light from LED 2, which serves as the second light source, may be visually presented. The ADC levels are associated with the light-receiving characteristic of a detector 1330 configured as a photo detector.

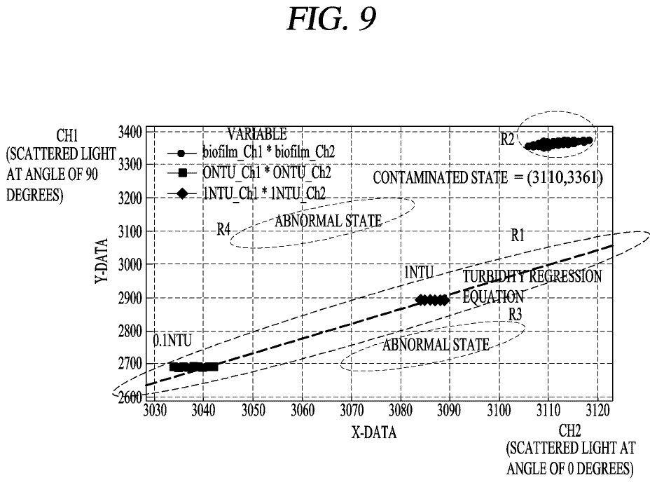

With reference to FIGS. 1 to 5, the ADC levels represent digital values that result from converting analog values of the first scattered light and the second reflected light, and may be output by the processor 1400. As the turbidity increases from 0 NTU to 1 NTU, the ADC level of the first scattered light resulting from first incident light from LED 1, which serves as the first light source 1310, may increase from 3300 to 3425. With reference to the ADC level of the first scattered light, the ADC level may correspond to a fourth region R4 that corresponds to a contaminated state in FIG. 9.

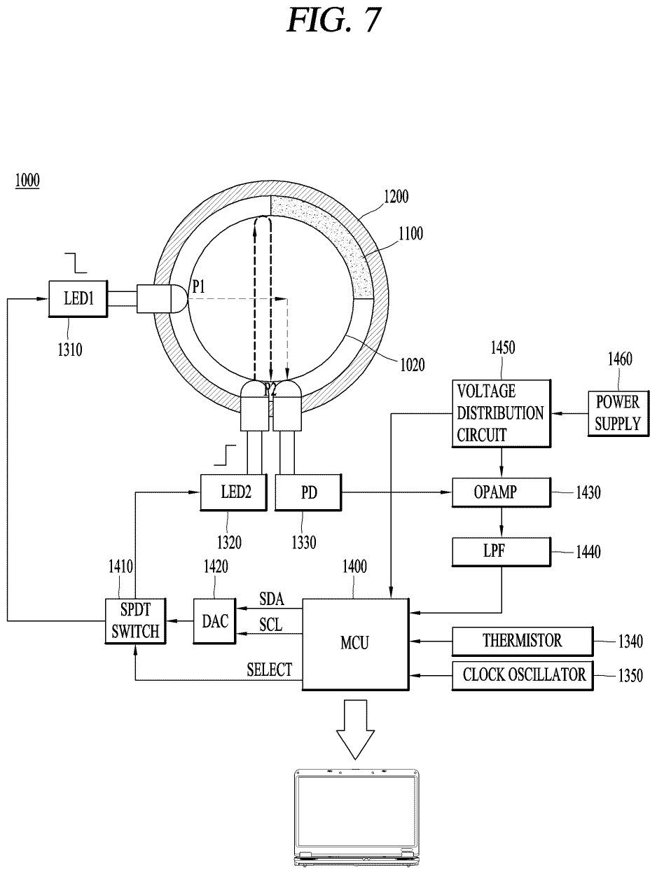

As the turbidity increases from 0 NTU to 1 NTU, the ADC level of the second reflected light resulting from second incident light from LED 2, which serves as the second light source 1320, falls within a predetermined value range starting at 3300. The ADC level of the second reflected light may fall within a different range that lies outside a range in FIG. 7. When the ADC level of the second reflected light deviates from that of the first reflected light, such deviation indicates a contamination state resulting from biofilms or the like, rather than being caused by foreign materials.

When the ADC level of the second reflected light deviates from the level of the ADC level of the first reflected light, this deviation may correspond to the fourth region R4 corresponding to the contaminated state in FIG. 9. The characteristics of the graph in FIG. 7 show changes in turbidity caused by water pollution. Other contamination characteristics may be identified by utilizing one-dimensional linear characteristics originating from LED 1, which serves as the first light source, and deviation characteristics originating from LED 2, which serves as the second light source.

With reference to (b) of FIG. 4 and FIG. 5, the adjustment of the intensity of the second light source by the reflector 1100b is described. As the reflector 1100b is arranged in one region of the first quadrant and one region of the second quadrant, the reflection ratio of third reflected light resulting from incident light from the second light source 1320 increases compared to that in a structure in (a) of FIG. 4. The degree of surface contamination may be determined by comparing the third reflected light resulting from the incident light from the second light source 1320 with the second reflected light resulting from contamination of the surface of the fluid channel pipe 1020.

Because the detector 1330 is arranged at the same location as the second light source 1320, the third reflected light, which results from the light propagating through the inside of the fluid channel pipe 1020 and being reflected at the fourth point P4 on the fluid channel pipe 1020, may have a low value. As the value of the third reflected light, which is reflected through the reflector 1100b, increases, the intensity of the second light source 1320 is adjustable to a level approximately equal to that of the first light source 1310.

When the intensity of the second light source 1320 is greater than that of the first light source 1310 by at least a threshold level and the turbidity increases due to increased foreign material accumulation, a turbidity difference between the first reflected light and the second reflected light may appear greater than that indicated by a slope in FIG. 5. When the intensity of the first light source 1310 is greater than that of the second light source 1320 by at least the threshold level, it cannot be determined whether an ADC level exceeding a normal level results from the contamination of the surface of the fluid channel pipe 1020 or from the increase in turbidity due to the increased foreign material accumulation. Therefore, using the second light source 1320 the intensity of which is adjusted, by the reflector 1100b, to a level approximately equal to the intensity of the first light source 1310, it can be determined whether the ADC level exceeding the normal level results from the contamination of the surface of the fluid channel pipe 1020 or from the increase in turbidity due to the increased foreign material accumulation.

In summary, the reflector 1100 in (a) of FIG. 4 is structured to increase a value of the first scattered light that is detected by the detector 1330. The reflector 1100 in (a) of FIG. 4 may be formed in a first sector shape spanning an angular extent of less than 90 degrees. For example, the reflector 1100 may be formed in the first sector shape spanning an angular extent of 60 degrees. The reflector 1100 can be formed in the first sector shape spanning an angular extent of 60 degrees, extending from 15 degrees to 75 degrees with respect to the X-axis.

The reflector 1100b in (b) of FIG. 4 is structured to increase the value of the third reflected light that is detected in a case where foreign materials are absent or present in a low ratio. The reflector 1100b in (b) FIG. 4 may be formed in a second sector shape spanning an angular extent of less than 90 degrees. For example, the reflector 1100b may be formed in a second sector shape of 60 degrees. The reflector 1100b may be formed in the first sector shape spanning an angular extent of 60 degrees, extending from 60 degrees to 120 degrees with respect to the X-axis.

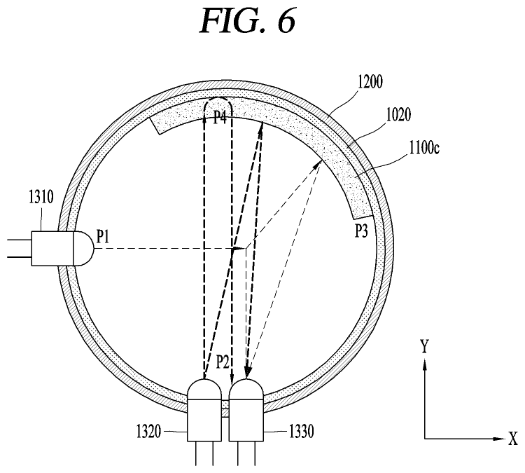

FIG. 6 is a view illustrating the structure of the fluid channel pipe in which the reflector is formed in the shape of a sector spanning an angular extent of more than 90 degrees along regions of the fluid channel pipe that correspond to the first and second quadrant of the XY plane. FIG. 6 illustrates that the reflector 1100 is arranged on the inside of the fluid channel pipe 1020, but it is not limited thereto and may also be arranged on the outside of the fluid channel pipe 1020 as in FIG. 2.

With reference to FIG. 6, a reflector 1100c is structured to increase the value of the first scattered light that is detected by the detector 1330. In addition, in the case where no foreign materials are detected or where the detected foreign materials are present in a ratio lower than a threshold ratio, the reflector 1100c may increase the value of the detected third reflected light.

The reflector 1100c is capable of improving the detection accuracy of foreign materials having scattering components by increasing the value of the first scattered light and also improving the detection accuracy of foreign materials having reflecting components by increasing the value of the third reflected light.

As the size of the reflector 1100c increases, light components scattered at the boundary region of the reflector 1100c may increase, or the asymmetry of a light path may increase slightly. Therefore, the reflector 1100c needs to be formed on an upper region of the fluid channel pipe 1020 such that an arc angle thereof is greater than 90 degrees and less than a threshold angle.

The reflector 1100c may be formed as a structure in which the reflector 1100 in (a) of FIG. 4 and the reflector 1100b in (b) of FIG. 4 are coupled. The reflector 1100 in (a) of FIG. 4 has the first sector shape spanning an angular extent of 60 degrees, extending from 15 degrees to 75 degrees with respect to the X-axis. The reflector 1100b in (b) of FIG. 4 has the second sector shape spanning an angular extent of 120 degrees, extending from 15 degrees to 60 degrees with respect to the X-axis. Therefore, the reflector 1100c may be formed in a third sector shape spanning an angular extent of 105 degrees, extending from 15 degrees to 120 degrees with respect to the X-axis.

The reflector 1100c may be formed in the third sector shape spanning an angular extent from more than 90 degrees to less than 120 degrees along a curved surface of the fluid channel pipe 1020, between the third point P3 and the first point P1 on the inside or outside of the fluid channel pipe. The third point P3 on the fluid channel pipe 1020 may be a point that is opposite the first point P1. The fourth point P3 on the fluid channel pipe 1020 may be a point that is opposite the second point P2.

The reflector 1100c may be formed as an asymmetrical structure that extends in opposite directions from the fourth point P4 along the inside or outside of the fluid channel pipe 1020. The reflector 1100c may be formed such that a first end thereof is adjacent to the third point P3 on the fluid channel pipe 1020.

The reflector 1100c may be formed such that the first end thereof is adjacent to the third point P3 on the fluid channel pipe 1020, with an angular extent of 30 degrees. The reflector 1100c may be formed such that a second end of spaced from the first point P1 on the fluid channel pipe 1020, with an angular extent of 45 degrees or more.

In this regard, since the detector 1330 is arranged at the second point P2 on the fluid channel pipe 1020 rather than at the fourth point P4, the reflectors 1100b and 1100c may be formed to include the fourth point P4 as in (b) of FIG. 4 and FIG. 5. Therefore, in the device 1000 for sensing water quality according to the present disclosure, two light sources and one detector may be optimally arranged on a lower region (one region) of the fluid channel pipe 1020 and a reflector may be optimally arranged on an upper region (the other region) thereof. Consequently, foreign materials having scattering and reflecting components may be effectively detected.

Specifically, the device 1000 for sensing water quality is structured such that two light sources, that is, the first light source 1310 and the second light source 1320, are arranged at the first point P1 and the second point P2, respectively, on the fluid channel pipe 1020 and that the detector 1330 is arranged at the second point P2 on the fluid channel pipe 1020. In this regard, the reflectors 1100, 1100b, and 1100c can be arranged in different forms on the upper region of the fluid channel pipe 1020.

The detailed structure of the device 1000 for sensing water quality according to the present disclosure is described. The device 1000 for sensing water quality may be configured to selectively emit light from the first light source 1310 and second light source 1320 that are different from each other. In this regard, FIG. 7 is a detailed block diagram illustrating the device for sensing water quality in FIG. 2. With reference to FIGS. 1 to 7, the device 1000 for sensing water quality according to the present disclosure is described.

The device 1000 for sensing water quality may be configured to further include a switch 1410. Light emission can be controlled by the switch 1410 such that light is selectively emitted from either the first light source 1310 or the second light source 1320. In this regard, the switch 1410 may be configured to control a first light emission state of the first light source 1310 and a second light emission state of the second light source 1320. The switch 1410 may be configured as a single pole double throw (SPDT) switch such that the first light source 1310 or the second light source 1320 emits light.

The processor 1400 may control the switch 1410 such that the first light source 1310 remains turned on, thereby allowing the first light to be emitted into the internal region of the optical fluid channel pipe 1020. The processor 1400 may control the detector 1330 to measure the first turbidity value by detecting the scattered light resulting from the first light. The processor 1400 may control the switch 1410 such that the second light source 1320 remains turned on, thereby allowing the second light to be emitted into the internal region of the fluid channel pipe 1020. The processor 1400 may control the detector 1330 to measure the second turbidity value by detecting the reflected light resulting from the second light.

The processor 1400 may compute a turbidity value for the internal region of the fluid channel pipe 1020 based on the measured first turbidity value and the measured second turbidity value. Therefore, turbidity and fluid channel contamination may be computed and detected, respectively, using a sensor detecting a signal for low turbidity at levels suitable for potable water (s 3 NTU), the sensor being configured to be included in the device 1000 for sensing water quality according to the present disclosure. NTU stands for Nephelometric Turbidity Unit. Turbidity refers to the degree of cloudiness in water caused by suspended particles and other materials. Specifically, the turbidity obtained by dissolving 2.5 mL of a turbidity standard stock solution containing hydrazine sulfate and hexamethylenetetramine in 1 L of distilled water is defined as 1 NTU.

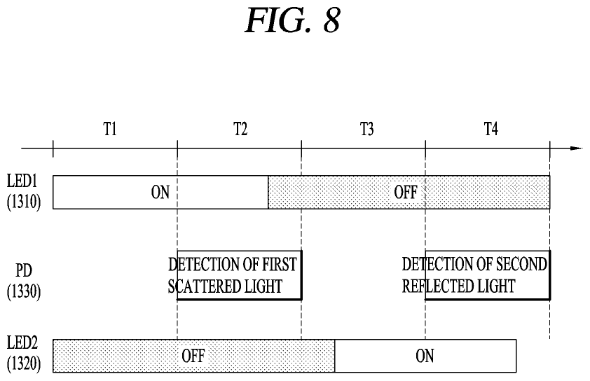

The device for sensing water quality according to the present disclosure may control the emission of light from the first and second light sources and detect the scattered light/reflected light by employing a time division scheme. In this regard, FIG. 8 is a conceptual diagram for controlling the emission of light from the first and second light sources and for detecting the first scattered light and the second reflected light by employing the time division scheme.

With reference to FIG. 8, the time division scheme may be employed to control the emission of light from the first and second light sources and to detect the first scattered light and the second reflected light. During a first time interval T1, the emission of light may be controlled with the first light source 1310 remaining turned on and the second light source 1320 remaining turned off. During a second time interval T1, subsequent to the first time interval T1, the first turbidity value may be measured by detecting the first scattered light resulting from the first incident light from the first light source 1310.

Before the start of a third time interval T3, the first light source 1310 may remain turned off. After the start of the third time interval T3, the second light source 1320 may remain turned on. Therefore, during the second time interval T1, only the first scattered light from the first light source 1310 may be detected. The propagation directions of the first incident light and the first scattered light from the first light source 1310 differ by an angle of 90 degrees, and thus, interference between them may be negligible.

During the third time interval T3, subsequent to the second time interval T2, the emission of light may be controlled with the first light source 1310 remaining turned on and the second light source 1320 remaining turned off. During a fourth time interval T4, subsequent to the third time interval T3, the second turbidity value may be measured by detecting the second reflected light from the second incident light from the second light source 1320. Therefore, during the fourth time interval T4, only the second reflected light from the second light source 1320 may be detected. The propagation directions of the second incident light and the second reflected light from the second light source 1320 vary by an angle of 180 degrees, and during the fourth time interval T4, the second reflected light is detected by the detector 1330. During the fourth time interval T4, the second incident light remains unreflected by foreign materials, and thus, the second incident light is not detected by the detector 1330.

A second duration during which the first light source 1310 remains turned off may be set to be longer than a first duration during which the first light source 1310 remains turned on. A third duration during which the second light source 1320 remains turned off may be set to be longer than a fourth duration during which the second light source 1320 remains turned on.

With reference to FIGS. 1 to 8, the device 1000 for sensing water quality, which controls the emission of light from the first and second light sources and detects the scattered light/reflected light by employing the time division scheme, is described. The processor 1400 may control the second light source 1320 to remain turned off during the first time interval T1, during which the first light source 1310 remains turned on. The processor 1400 may perform control such that the first turbidity value during the second time interval T2, subsequent to the first time interval T1. For a predetermined time during the second time interval T2, the first light source 1310 may remain turned on, and before the start of the third time interval T3, the first light source 1310 may remain turned off, After the start of the third time interval T3, the second light source 1320 may remain turned on.

The processor 1400 may control the first light source 1310 to remain turned off during the third time interval, during which the second light source 1320 remains turned on. In this regard, the first light source 1310 may remain turned off after the start of the third time interval. The processor 1400 may perform control such that the second turbidity value is measured during the fourth time interval, subsequent to the third time interval. For a predetermined time during the fourth time interval, the second light source 1320 may remain turned on.

The device 1000 for sensing water quality may include a plurality of constituent elements such that signals are converted, amplified, and processed to compute the turbidity values. In this regard, the device 1000 for sensing water quality may be configured to further include a digital-to-analog converter (DAC) 1420, an operational amplifier 1430, and a low-pass filter (LPF) 1440.

The digital-to-analog converter 1420 may be arranged between the processor 1400 and the switch 1410. The digital-to-analog converter 1420 may receive a serial data signal (SDC) and a serial clock signal (SCL) from the processor 1400. The digital-to-analog converter 1420 may be configured to transfer control signals for controlling the first light source 1310 and the second light source 1320, through the switch 1410.

The operational amplifier 1430 may be connected to the detector 1330 and configured to amplify signals associated with the detected scattered light resulting from the first light and the detected reflected light resulting from the second light. The low-pass filter 1440 may be configured to filter the amplified signals such that low-frequency signal components are passed through and that the passed low-frequency signal components are transferred to the processor 1400.

The device 1000 for sensing water quality may be configured to further include a voltage distribution circuit 1450 and a power supply 1460. The voltage supplied from the power supply 1460 through the voltage distribution circuit 1450 may be converted into operating voltages to operate the processor 1400 and the operational amplifier 1430.

When it is determined that the fluid channel pipe 1020 is contaminated with condensation based on the first turbidity value, obtained using the first light, and the second turbidity value, obtained using the second light, the device 1000 for sensing water quality may perform temperature control. The device 1000 for sensing water quality may include a thermistor 1340 for temperature measurement. In addition, the device 1000 for sensing water quality may include a clock oscillator 1350 to generate control signals associated with timing for controlling the first light source 1310 and the second light source 1320.

As described above, the device 1000 for sensing water quality may be configured to further include the thermistor 1340 and the clock oscillator 1350. The thermistor 1340 may be operatively coupled to the processor 1400. The thermistor 1340 may be configured to measure the temperature of the internal region of the fluid channel pipe 1020.

In this regard, the processor 1400 may determine whether the first turbidity value, based on the scattered light resulting from the first light, or the second turbidity value, based on the reflected light resulting from the second light, exceeds a threshold value. When the first turbidity value or the second turbidity value exceeds the threshold value, the processor 1400 may determine whether the fluid channel pipe 1020 is contaminated with condensation. When it is determined that the fluid channel pipe 1020 is contaminated with condensation, the processor 1400 may control a temperature adjustment device such that the temperature of the internal region of the fluid channel pipe 1020 is controlled.

When the first turbidity value and the second turbidity value are less than the threshold value, the processor 1400 may determine whether the first turbidity value and the second turbidity value are indicative of an abnormal or normal condition. In this regard, FIG. 9 is a graph that is referenced to determine a water quality state by distinguishing between two-dimensional turbidity patterns derived from the first turbidity value and the second turbidity value.

With reference to FIG. 9, the first turbidity value and the second turbidity value may be mapped to coordinates on the X-axis and the Y-axis, respectively. In this regard, when a liquid is in a normal state of low turbidity, the first turbidity value and the second turbidity value vary according to a turbidity regression equation defined in the form of a linear function as in the first region R1 in FIG. 5, and characteristic turbidity values of water quality change.

When the first and second turbidity values follow the turbidity regression equation defined in the form of a linear function, it may be determined that the inside of the conduit remains normal. When the first and second turbidity values do not follow the turbidity regression equation defined in the form of a linear function, it may be determined that the second region R2 and the third region R3 remain abnormal or that the fourth region R4 remains contaminated. In this regard, an abnormal state of water quality can be detected by distinguishing between two turbidity patterns based on machine learning.

A high-performance low-turbidity sensor that uses a machine learning technique is configured to have an optical path variable composite structure employing LED 1 and LED 2 that serve as the first and second light sources, respectively. The first and second light sources refer to structures for detecting the scattered light and the reflected light in water, respectively. These structures are employed to overcome limitations in addressing environmental factors, such as the detection of foreign materials, which are associated with existing simple techniques relying on scattered-light sensing. Under conditions such as condensation in the fluid channel pipe, foreign material contamination, and biofilm formation, it is difficult to distinguish between general water-quality contamination and contamination of the fluid channel pipe when relying solely on the detection of the scattered light. Therefore, to determine either the contamination of the surface of the fluid channel pipe or the water-quality contamination, it is necessary to analyze the characteristics of the scattered light and perform composite detection using the reflected light or the absorbed light.

With reference to FIGS. 1 to 9, a composite detection method for determining the water quality contamination, distinguished from the contamination of the surface of the fluid channel pipe 1020, is described. To implement the above-mentioned method, the scattered light is detected by arranging the first light source 1310 spaced from the detector 1330, which serves as a light-receiving part, with an angular separation of 90 degrees. The absorbed light or the reflected light may be detected by arranging the second light source 1320 at the same point as the detector 1330, which serves as the light-receiving part, with an angular extent of 0 degrees. The first light source 1310 and the second light source 1320 operate in an alternating manner. When the first light source 1310 remains turned on, the second light source 1320 remains turned off, and when the first light source 1310 remains turned off, the second light source 1320 remains turned on.

The scattered light, which corresponds to a condition of low turbidity, is detected using the first light source 1310, and under the condition that light is emitted from the second light source 1320, a determination is made as to whether a value of the light received from the first light source 1310 is valid. When the value of the received light is determined to be abnormal rather than corresponding to pure water quality contamination, either a condensation phenomenon or the contamination of the fluid channel pipe is identified based on predetermined conditions. When the condensation phenomenon is identified, temperature adjustment is required. When the contamination of the fluid channel pipe is identified, re-measurement is required to be performed through fluid-channel cleaning control. Consequently, only the level of contamination associated with water quality may be measured.

With reference to FIG. 9 and Expression 1 below, a first detection value (TLED1(x)), which is obtained from the value of the received light according to the turbidity (X) determined using the first light source, may be expressed as a linear function. A second detection value (TLED2(x)), which is obtained from the value of the received light according to the turbidity (X) determined using the second light source, may be expressed as a linear function. The first detection value (TLED1(x)) may be expressed using the second detection value (TLED2(x)) and the coefficients of a linear function.

$$TLED 1 ( x ) = aX + b \qquad Equation 1$$

$$TLED 2 ( x ) = cX + d$$

$$TLED 1 ( x ) = a ( TLED 2 ( x ) - d ) / c + b$$

In Equation 1, when the linear relationship, defined in the form of a linear function, between TLED1(x) and TLED2(x) is satisfied, the baseline turbidity of the water quality is inferred. Conversely, satisfaction of the inequality condition indicates the existence of abnormal values due to environmental contamination is indicated, thereby requiring re-measurement through corrective processing.

In a case where foreign materials, such as biofilms, are present within the fluid channel pipe, values deviating from the linear regression equation are output, indicating that such values do not represent the characteristic turbidity values of the water quality. Therefore, as in the fourth region R4, the first turbidity value and the second turbidity value that deviate from the turbidity regression equation defined in the form of a linear function may be detected. In this regard, the first turbidity value may be detected to be greater than the second turbidity value. For example, the first turbidity value and the second turbidity value may be detected as 3110 and 3361, respectively. Therefore, when foreign materials are removed through the fluid-channel cleaning control, the first turbidity value and the second turbidity value vary according to the turbidity regression equation defined in the form of a linear function, as in the first area R1. Accordingly, the normal regression model in the form of a linear function is employed, thereby enabling the detection of normal water quality turbidity.

With reference to FIGS. 1 to 9, the water quality sensor device 1000 according to the present disclosure is described. When the first turbidity value and the second turbidity value are less than the threshold value, the processor 1400 may determine whether the first turbidity value and the second turbidity value remain abnormal or normal. In this regard, the threshold value for the first turbidity value or the second turbidity value may be set to 4,000. However, the threshold value for the first turbidity value or the second turbidity value is not limited to 4,000 and may be adjusted depending on the intended application. The first turbidity value and the second turbidity value are values that result from digitally converting first voltage value and a second voltage value, respectively, that are detected by the detector 1330. For example, a voltage value of up to 1.8 V may be detected and converted into a 12-bit digital value. Therefore, the value resulting from digitally converting a voltage value of up to 1.8 V corresponds to 4096.

The processor 1400 may determine whether the first turbidity value and the second turbidity value remain in a normal state where a linear relationship defined in the form of a linear function is established, or in an abnormal state that deviates from the linear relationship. When the first turbidity value and the second turbidity value remain normal, the processor 1400 may compute the turbidity value for the internal region of the fluid channel pipe 1020 based on the first turbidity value and the second turbidity value. The normal state may be defined as the first region R1 on the XY plane, where the first turbidity value and the second turbidity value form the linear relationship defined in the form of a linear function, with the first turbidity value and the second turbidity values as variables.

When the first turbidity value and the second turbidity value remain abnormal, the processor 1400 may determine whether the fluid channel pipe 1020 is contaminated with foreign materials in the internal region thereof or with biofilms. The abnormal state may be defined as the second region R2 on the XY plane, where the first turbidity value is greater than in the first region R1, or as the third region R3 on the XY plane, where the second turbidity value is greater than in the first region R1, with the first turbidity value and the second turbidity values as variables.

When it is determined that the contaminated state is caused by foreign materials in the internal region of the fluid channel pipe 1020 or by biofilms, the processor 1400 may perform control such that a fluid channel of the fluid channel pipe 1020 is cleaned. In this regard, the contaminated state may be defined as the fourth region R4 on the XY plane, where the first turbidity value and the second turbidity value are greater than those associated with an abnormal state of the third region, with the first turbidity value and the second turbidity values as variables. The processor 1400 may perform control such that water is introduced into the internal region of the fluid channel pipe 1020 in a state where the fluid channel of the fluid channel pipe 1020 is cleaned.

The water quality control device 1000 is capable of computing a turbidity value based on the first and second turbidity values under a controlled temperature condition when the fluid channel pipe 1020 is contaminated with condensation. In this regard, the processor 1400 may control the switch 1410 such that the first light source 1310 remains turned on to emit the first light into the internal region of the fluid channel pipe 1020 under the controlled temperature condition. The processor 1400 may measure the first turbidity value by detecting the scattered light resulting from the first light while the first light source 1310 remains turned on.

The processor 1400 may control the switch 1410 such that the second light source 1320 remains turned on to emit the second light into the internal region of the fluid channel pipe 1020 under the controlled temperature condition. The processor 1400 may measure the second turbidity value by detecting the reflected light resulting from the second light. When the measured first turbidity value or the measured second turbidity value remains normal, the processor 1400 may compute the turbidity value for the internal region of the fluid channel pipe 1020 based on the first turbidity value and the second turbidity value.

When the first turbidity value and the second turbidity value are less than the threshold value, the processor 1400 may determine whether the fluid channel pipe 1020 is contaminated with foreign materials in the internal region thereof or with biofilms. When it is determined that the fluid channel pipe 1020 is contaminated with foreign materials in the internal region thereof or with biofilms, the processor 1400 may perform control such that the fluid channel of the fluid channel pipe 1020 is cleaned. The processor 1400 may perform control such that water is introduced into the internal region of the fluid channel pipe 1020 in the state where the fluid channel of the fluid channel pipe 1020 is cleaned.

The processor 1400 may control the switch 1410 such that the first light source 1310 remains turned on to emit the first light into the internal region of the fluid channel pipe 1020 in a state where water is introduced into the internal region of the fluid channel pipe 1020. The processor 1400 may measure the first turbidity value by detecting the scattered light resulting from the first light while the first light source 1310 remains turned on.

The processor 1400 may control the switch 1410 such that the second light source 1320 remains turned on to emit the second light into the internal region of the fluid channel pipe 1020 in the state where water is introduced into the internal region of the fluid channel pipe 1020. The processor 1400 may measure the second turbidity value by detecting the reflected light resulting from the second light while the second light source 1320 remains turned on. When the measured first turbidity value or the measured second turbidity value remains normal, the processor 1400 may compute the turbidity value for the internal region of the fluid channel pipe 1020 based on the first turbidity value and the second turbidity value.

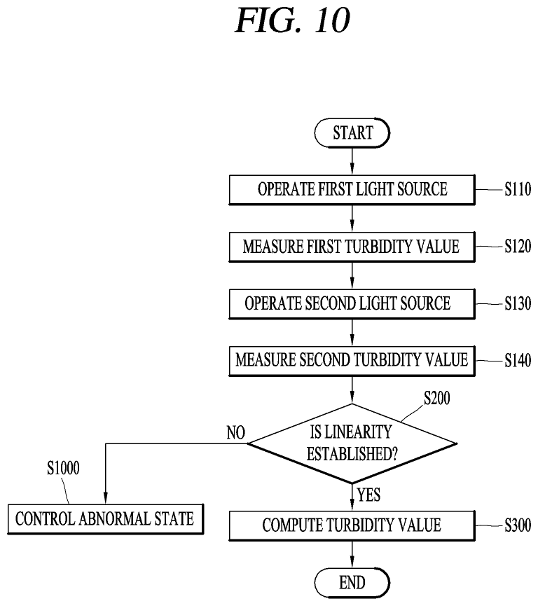

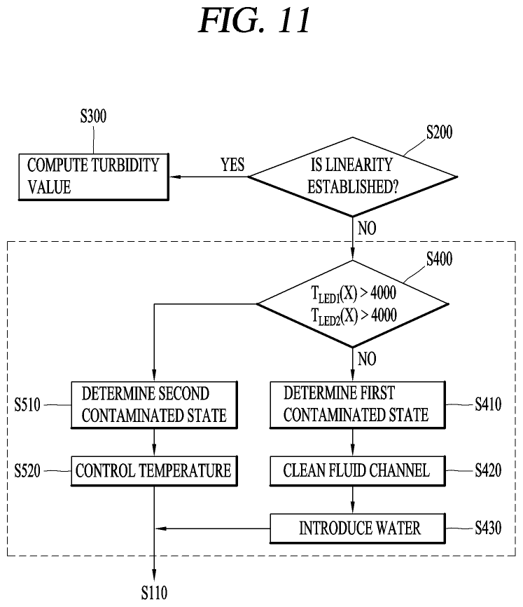

The device 1000 for sensing water quality according to one aspect of the present disclosure has been described above. A method of sensing water quality according to another aspect of the present disclosure is described below. In this regard, FIGS. 10 and 11 are views illustrating the method of sensing water quality according to the aspect of the present disclosure. The method of sensing water quality in FIGS. 10 and 11 may be performed by the processor of the device for sensing water quality in FIGS. 1 to 6. The above-described configuration and operation of the device 1000 for sensing water quality can apply to the following method of sensing quality.

With reference to FIGS. 1 to 11, the method of sensing water quality according to the present disclosure is described. The method of sensing water quality may be performed by the processor 1400 of the device 1000 for sensing water quality. The method of sensing water quality may include Step S110 of operating the first light source, Step S120 of measuring the first turbidity value, Step S130 of operating the second light source, Step S140 of measuring the second turbidity value, and Step S300 of computing the turbidity value.

In Step S110 of operating the first light source, the switch may be switched such that the first light source arranged at the first point remains turned on to emit the first light into the internal region of the fluid channel pipe. In Step S120 of measuring the first turbidity value, the first turbidity value may be measured by enabling the detector to detect the scattered light resulting from the first light being scattered within the internal region of the fluid channel pipe.

In Step S130 of operating the second light source, the switch may be switched such that the second light source arranged at the second point remains turned on to emit the second light into the internal region of the fluid channel pipe. In Step S140 of measuring the second turbidity value, the second turbidity value may be measured by enabling the detector to detect the reflected light resulting from the second light being reflected by the surface of the fluid channel pipe. In Step S300 of computing the turbidity value, the turbidity value for the internal region of the fluid channel pipe may be computed based on the first turbidity value and the second turbidity value.

In Step S110 of operating the first light source, the second light source may be controlled to remain turned off during the first time interval during which the first light source remains turned on. In Step S120 of measuring the first turbidity value, the first turbidity value may be measured during the second time interval, subsequent to the first time interval. In Step S130 of operating the second light source, the first light source may be controlled to remain turned off during the third time interval during the second light source remains turned on. In Step S140 of measuring the second turbidity value, the second turbidity value may be measured during the fourth time interval, subsequent to the third time interval.

The method of sensing water quality may be configured to further include Step S200 of determining linearity. In Step S200 of determining the linearity, it may be determined whether the first turbidity value and the second turbidity value remain in the normal state where the linear relationship defined in the form of a linear function is established, or in the abnormal state that deviates from the linear relationship. The method of sensing water quality may be configured to perform Step S1000 of controlling the abnormal state when the first turbidity value and the second turbidity value remain abnormal.

The method of sensing water quality may be configured to further include Step S400 of comparing with the threshold value. In the method of sensing water quality, Step S1000 of controlling the abnormal state may be performed in Step S400 of comparing with the threshold value. Specifically, when the first turbidity value and the second turbidity value remain abnormal, Step S400 of comparing with the threshold value may be performed. In Step S400 of comparing with the threshold value, it may be determined whether the first turbidity value or the second turbidity value exceeds the threshold value in the abnormal state.

The method of water quality sensing may include Step S410 of determining a first contaminated state, Step S420 of cleaning the fluid channel, and Step S430 of introducing water. The method of sensing water quality may include Step S510 of determining a second contaminated state and Step S520 of controlling temperature.

When the first turbidity value and the second turbidity value do not exceed the threshold value, Step S410 of determining the first contaminated state may be performed. In Step S410 of determining the first contaminated state, it may be determined whether the fluid channel pipe is contaminated with foreign materials in the internal region thereof or with biofilms. When it is determined that the fluid channel pipe is contaminated with foreign materials in the internal region thereof or with biofilms, Step S420 of cleaning the fluid channel may be performed. When the first turbidity value and the second turbidity value are less than the threshold value, in Step S420 of cleaning the fluid channel, control may be performed such that the fluid channel of the fluid channel pipe is cleaned. Step S430 of introducing water may be performed in a state where the fluid channel of the fluid channel pipe is cleaned. In Step S430 of introducing water, control may be performed such that water is introduced into the internal region of the fluid channel pipe.

When it is determined, in Step S200 of determining the linearity, that the first turbidity value and the second turbidity value remain normal, Step S300 of computing the turbidity value may be performed. In Step S300 of computing the turbidity value, the turbidity value for the internal region of the fluid channel pipe may be computed based on the first turbidity value and the second turbidity value.

When the first turbidity value or the second turbidity value exceeds the threshold value in Step S400 of comparing with the threshold value, Step S510 of determining the second contaminated state may be performed. In Step S510 of determining the second contaminated state, it may be determined whether the fluid channel pipe is contaminated with foreign materials in the internal region thereof or with biofilms. When it is determined that the fluid channel pipe is contaminated with condensation, Step S520 of controlling temperature may be performed. When the first turbidity value or the second turbidity value exceeds the threshold value, in Step S520 of controlling temperature, the temperature adjustment device coupled to the processor may be controlled such that the temperature of the internal region of the fluid channel pipe is controlled.

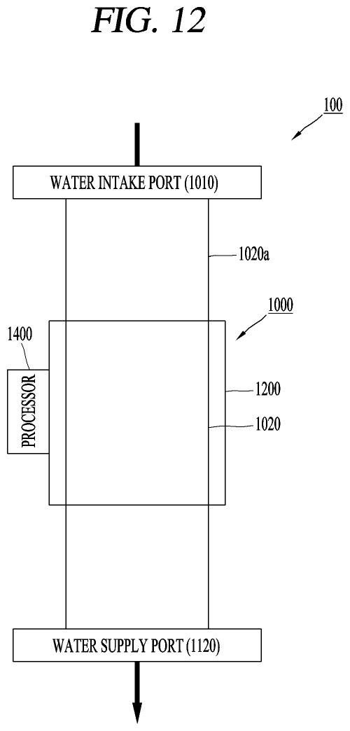

The device for sensing water quality and the method of sensing water quality according to the present disclosure have been described above. An apparatus for purifying water according to still another aspect of the present disclosure, which includes the device for sensing water quality, is described below. In this regard, FIG. 12 is a view illustrating the configuration of the apparatus for purifying water according to the aspect of the present disclosure, which includes the device for sensing water quality. The above-described configuration and operation descriptions of the device 1000 for sensing water quality and the method of sensing water quality may apply to an apparatus 100 for purifying water, which includes the device 1000 for sensing water quality. The device 1000 for sensing water quality of FIG. 12 may find application in household appliances other than the apparatus 100 for purifying water, such as washing machines.

With reference to FIG. 12, the apparatus 100 for purifying water may be configured to include a water intake port 1010, the device 1000 for sensing water quality, and a water supply port 1030. With reference to FIGS. 1 to 12, the apparatus 100 for purifying water according to the aspect of the present disclosure, which includes the device 1000 for sensing water quality, is described. The water intake port 1010 may be formed such that water is introduced therethrough. The device 1000 for sensing water quality may be configured to detect the turbidity of water flowing through the fluid channel pipe 1020 from the water intake port 1010. The device 1000 for sensing water quality may be arranged in one portion of a pipe 1020a in which the water intake port 1010 and the water supply outlet 1030 are formed. The water supply outlet 1030 may be formed such that water, the turbidity of which is detected, is discharged therethrough.

The device 1000 for sensing water quality may be configured to include the fluid channel pipe 1020, the external light blocking structure 1200, the first light source 1310, the second light source 1320, and the detector 1330. The device 1000 for sensing water quality may be configured to include the processor 1400 or to operate in conjunction with the processor 1400.

The external light blocking structure 1200 may be formed such that the fluid channel pipe 1020 is inserted into the external light blocking structure 1200. The external light blocking structure 1200 may be configured such that the inlet and outlet of the fluid channel pipe 1020 are exposed at the first lateral surface and the second lateral surface, respectively. A liquid, such as water, may be accommodated within the fluid channel pipe 1020. The external light blocking structure 1200 may be formed to surround the outside of the fluid channel pipe 1020.

The first light source 1310 may be arranged at to the first point on the fluid channel pipe 1020. The first light source 1310 may be configured such that the first light is emitted into the internal region of the fluid channel pipe 1020. The second light source 1320 may be arranged at the second point on the fluid channel pipe 1020. The second light source 1320 may be configured such that the second light is emitted into the internal region of the fluid channel pipe 1020.

The detector 1330 may be configured to detect the scattered light resulting from the first light being scattered within the internal region of the fluid channel pipe 1020. In addition, the detector 1330 may be configured to detect the reflected light resulting from the second light being reflected by the surface of the fluid channel pipe 1020. The processor 1400 may be operatively coupled to the first light source 1310, the second light source 1320, and the detector 1330. The processor 1400 may be configured to measure the turbidity of a liquid within the fluid channel pipe 1020.

The device 1000 for sensing water quality may be configured to further include the switch 1410 such that light emission is performed to selectively enable one of the first and second light sources 1310 and 1320 to emit light. In this regard, the switch 1410 may be configured to control the first light emission state of the first light source 1310 and the second light emission state of the second light source 1320. The switch 1410 may be configured as a single pole double throw (SPDT) switch such that the first light source 1310 or the second light source 1320 emits light.

The processor 1400 may control the detector 1330 to measure the first turbidity value by detecting the scattered light resulting from the first light. The processor 1400 may control the detector 1330 to measure the first turbidity value by detecting the scattered light resulting from the first light. The processor 1400 may control the switch 1410 such that the second light source 1320 remains turned on, thereby allowing the second light to be emitted into the internal region of the fluid channel pipe 1020. The processor 1400 may control the detector 1330 to measure the second turbidity value by detecting the reflected light resulting from the second light. The processor 1400 may compute the turbidity value for the internal region of the fluid channel pipe 1020 based on the measured first turbidity value and the measured second turbidity value.

The processor 1400 may measure the first turbidity value by enabling the detector 1330 to detect the scattered light resulting from the first light. The processor 1400 may measure the second turbidity value by enabling the detector 1330 to detect the reflected light resulting from the second light. The processor 1400 may compute the turbidity value for the internal region of the fluid channel pipe 1020 based on the first turbidity value and the second turbidity value. It may be detected whether the fluid channel pipe 1020 is contaminated. The processor 1400 may compute the turbidity value for the internal region of the fluid channel pipe 1020 based on the first turbidity value associated with the measured scattered light resulting from the first light and on the detected reflected light resulting from the second light. It may be detected whether the fluid channel pipe 1020 is contaminated.

The processor 1400 may determine whether the first turbidity value and the second turbidity value remain abnormal or normal. The processor 1400 may determine whether the first turbidity value and the second turbidity value remain in the normal state where the linear relationship defined in the form of a linear function is established, or in the abnormal state that deviates from the linear relationship. When the first turbidity value and the second turbidity value remain normal, the processor 1400 may compute the turbidity value for the internal region of the fluid channel pipe 1020 based on the first turbidity value and the second turbidity value.

When the first turbidity value and the second turbidity value are less than the threshold value in the abnormal state, the processor 1400 may perform control such that the fluid channel of the fluid channel pipe 1020 is cleaned. When the first turbidity value and the second turbidity value remain abnormal, the processor 1400 may determine whether the fluid channel pipe 1020 is contaminated with foreign materials in the internal region thereof or with biofilms. When it is determined that the fluid channel pipe 1020 is contaminated with foreign materials in the internal region thereof or with biofilms, the processor 1400 may perform control such that the fluid channel of the fluid channel pipe 1020 is cleaned. In addition, the processor 1400 may perform control such that water is introduced into the internal region of the fluid channel pipe 1020 in the state where the flow channel in the fluid channel pipe 1020 is cleaned.

When the first turbidity value or the second turbidity value exceeds the threshold value in the abnormal state, the processor 1400 may control the temperature adjustment device coupled to the processor 1400 such that the temperature of the internal region of the fluid channel pipe 1020 is controlled. When it is determined that the fluid channel pipe 1020 is contaminated with condensation based on the first turbidity value, obtained using the first light, and on the second turbidity value, obtained using the second light, the device 1000 for sensing water quality may perform temperature control.

In this regard, the processor 1400 may determine whether the first turbidity value, based on the scattered light resulting from the first light, or the second turbidity value, based on the reflected light resulting from the second light, exceeds the threshold value. When the first turbidity value or the second turbidity value exceeds the threshold value, the processor 1400 may determine whether the fluid channel pipe 1020 is contaminated with condensation. When it is determined that the fluid channel pipe 1020 is contaminated with condensation, the processor 1400 may control the temperature adjustment device such that the temperature of the internal area of the fluid channel pipe 1020 is controlled.

The device for sensing water quality, the method of sensing water quality, and household appliances, such as apparatus for purifying water, which includes the device for sensing water quality, according to the present disclosure, have been described above. The technical effects of a device for sensing water quality, a method of sensing water quality, and household appliances, such as an apparatus for purifying water, which includes the device for sensing water quality, according to the present disclosure, are described below.

According to the present disclosure, there is provided a device for measuring water quality, the device being capable of performing a sensing function that detects the quality of low-turbidity water using scattered light and reflected light that result from the scattering and reflection of incident light by foreign materials.

According to the present disclosure, water turbidity can be measured in real time by preventing a time delay phenomenon when estimating the water turbidity caused by foreign materials in a fluid, using the scattered light and the reflected light that result from the scattering and reflection of the incident light by the foreign materials.

According to the present disclosure, foreign materials having scattering and reflecting components can be effectively detected by optimally arranging two light sources and one detector in one region of a fluid channel pipe and optimally arranging a reflector on the other region thereof.

According to the present disclosure, an abnormal state of water quality and a contaminated state of water can be effectively determined through a two turbidity pattern resulting from the scattered light and the reflected light.

According to the present disclosure, erroneous detection issues, caused by water quality conditions such as biofilms and activated carbon in existing sensors, can be overcome in a low-turbidity region applicable to household drinking water appliances by identifying a machine-learning-based two-dimensional turbidity pattern.

According to the present disclosure, the turbidity of water in household drinking water appliances can be detected, thereby contributing to the safety of potable water. The safety of potable water can be ensured by preventing erroneous detection caused by contamination of a fluid channel and detecting the turbidity of water.

According to the present disclosure, a customized filter replacement cycle can be set based on contamination standards by preventing erroneous detection caused by the contamination of the fluid channel and detecting the turbidity of water. As a result, unnecessary replacement costs can be reduced.

According to the present disclosure, a cost-effective device for detecting water quality can be manufactured using low-cost light-emitting diodes and photodiodes, thereby expanding its applications in household appliances.

According to the present disclosure, there is provided a household appliance, such as an apparatus for purifying water, which is capable of detecting low-turbidity water and measuring the turbidity of water in real time by employing an optimal structure including two light sources and one detector.

According to the present disclosure, a household appliance, such as an apparatus for purifying water, can provide a service for maintaining customer satisfaction by monitoring and measuring water quality in real time, thereby controlling the water quality accordingly.

The detailed description should not be interpreted as limiting in any respect but rather should be considered as illustrative. The scope of the present disclosure should be determined by the proper construction of the following claims. All equivalent modifications to the embodiments of the present disclosure fall within the scope of the present disclosure.

What is claimed is:

1. A device for sensing water quality, the device comprising:
a fluid channel pipe;
an external light blocking structure formed such that the external light blocking structure surrounds the fluid channel pipe and that the fluid channel pipe is inserted into the external light blocking structure, and configured such that an inlet and an outlet of the fluid channel pipe are exposed at a first lateral surface and a second lateral surface, respectively;
a first light source arranged at a first point on the fluid channel pipe and configured to emit first light into an internal region of the fluid channel pipe;
a second light source arranged at a second point on the fluid channel pipe and configured to emit second light into the internal region of the fluid channel pipe;
a detector configured to detect scattered light resulting from the first light being scattered within the internal region of the fluid channel pipe, and to detect reflected light resulting from the second light being reflected by a surface of the fluid channel pipe; and
a processor operatively coupled to the first light source, the second light source, and the detector, and configured to measure the turbidity for a liquid within the fluid channel pipe based on the detected scattered light resulting from the first light and the detected reflected light resulting from the second light, and to detect whether the fluid channel pipe is contaminated.

2. The device of claim 1, wherein the first point at which the first light source is arranged and a point at which the detector is arranged are spaced apart with an angular extent of 90 degrees, and
wherein the second point at which the second light source is arranged and the point at which the detector is arranged are positioned at an arc angle of 0 degrees.

3. The device of claim 1, further comprising:
a reflector formed along the inside or outside of the fluid channel pipe between a third point and a fourth point on the inside or outside thereof,
wherein the third point on the fluid channel pipe is a point that is opposite the first point on the fluid channel pipe,
wherein the fourth point on the fluid channel pipe is a point that is opposite the second point on the fluid channel pipe, and
wherein the reflector is formed in a first sector shape spanning an angular extent of 90 degrees or less along a curved surface of the fluid channel pipe between the third point and the fourth point on the fluid channel pipe.

4. The device of claim 1, further comprising:
a reflector formed in opposite directions from the fourth point along the inside or outside of the fluid channel pipe,
wherein the fourth point on the fluid channel pipe is a point that is opposite the second point on the fluid channel pipe, and
wherein the reflector is formed in a second sector shape spanning an angular extent of 90 degrees or less along a curved surface of the fluid channel pipe, the reflector being centered at the fourth point.

5. The device of claim 1, further comprising:
a reflector formed in a third sector shape spanning an angular extent from more than 90 degrees and to less than 120 degrees along a curved surface of the fluid channel pipe between the third point and the first point on the inside or outside of the fluid channel pipe,
wherein the reflector is formed as an asymmetrical structure that extends in opposite directions from the fourth point along the inside or outside of the fluid channel pipe,
wherein the third point on the fluid channel pipe is a point that is opposite the first point on the fluid channel pipe,
wherein the fourth point on the fluid channel pipe is a point that is opposite the second point on the fluid channel pipe, and
wherein the reflector is formed such that a first end thereof is adjacent to the third point and that a second thereof is spaced from the first point, with an angular extent of 45 degrees or more.

6. The device of claim 2, further comprising:
a switch configured to control a first light emission state of the first light source and a second light emission state of the second light source,
wherein the processor
controls the switch such that the first light source remains turned on to emit the first light into the internal region,
measures a first turbidity value by detecting the scattered light resulting from the first light,
controls the switch such that the second light source remains turned on to emit the second light into the internal region,
measures a second turbidity value by detecting the reflected light resulting from the second light, and
computes a turbidity value for the internal region based on the first turbidity value and the second turbidity value.

7. The device of claim 6, wherein the processor
controls the second light source to remain turned off during a first time interval during which the first light source remains turned on,
measures the first turbidity value during a second time interval, subsequent to the first time interval,
controls the first light source to remain turned on during a third time interval during which the second light source remains turned on, and
measures the second turbidity value during a fourth time interval, subsequent to the third time interval.

8. The device of claim 6, further comprising:
a digital-to-analog converter that is arranged between the processor and the switch, receives a serial data signal and a serial clock signal from the processor, and transfers control signals for controlling the first light source and the second light source, through the switch;
an operational amplifier connected to the detector and configured to amplify signals associated with the detected scattered light and the detected reflected light; and
a low-pass filter configured to filter the amplified signals such that low-frequency signal components are passed through and that the passed low-frequency signal components are transferred to the processor.

9. The device of claim 6, further comprising:
a thermistor coupled to the processor and configured to measure the temperature of the internal region of the fluid channel pipe,
wherein the processor
determines whether the first turbidity value or the second turbidity value exceeds a threshold value,
determines whether the fluid channel pipe is contaminated with condensation, when the first turbidity value or the second turbidity value exceeds the threshold value, and
controls a temperature adjustment device such that the temperature of the internal region of the fluid channel pipe is controlled, when it is determined that the fluid channel pipe is contaminated with the condensation.

10. The device of claim 9, wherein when the first turbidity value and the second turbidity value are less than the threshold value, the processor determines whether the first turbidity value and the second turbidity value remain in a normal state where a linear relationship defined in the form of a linear function is established, or in an abnormal state that deviates from the linear relationship, and
wherein when the first turbidity value and the second turbidity value remain normal, the processor computes the turbidity value for the internal region based on the first turbidity value and the second turbidity value.

11. The device of claim 10, wherein when the first turbidity value and the second turbidity value remain abnormal, the processor
determines whether the fluid channel pipe is contaminated with foreign materials in the internal region thereof or with biofilms,
performs control such that the fluid channel pipe is cleaned, when it is determined that a fluid channel of the fluid channel pipe is in a contaminated state caused by the foreign materials in the internal region thereof or by the biofilms, and
performs control such that water is introduced into the internal region of the fluid channel pipe, in a state where the fluid channel of the fluid channel pipe is cleaned.

12. The device of claim 11, wherein the threshold value for the first turbidity value or the second turbidity value is set to 4,000,
wherein the normal state is defined as a first region where the first turbidity value and the second turbidity value form the linear relationship defined in the form of a linear function,
wherein the normal state is defined as a second region where the first turbidity value is greater than in the first region, or as a third region where the second turbidity value is greater than in the first region, and
wherein the contaminated state is defined as a fourth region where the first turbidity value and the second turbidity value are greater than those associated with the abnormal state of the third region.

13. The device of claim 10, wherein in a state where the temperature of the internal region is controlled, the processor
controls the switch such that the first light source remains turned on to emit the first light into the internal region,
measures the first turbidity value by detecting the scattered light resulting from the first light,
controls the switch such that the second light source remains turned on to emit the second light into the internal region,
measures the second turbidity value by detecting the reflected light resulting from the second light, and
computes the turbidity value for the internal region based on the first turbidity value and the second turbidity value, when the measured first turbidity value or the measured second turbidity value remains normal.

14. The device of claim 10, wherein when the first turbidity value and the second turbidity value are less than the threshold value, the processor
determines whether the fluid channel pipe is contaminated with foreign materials in the internal region or with biofilms,
performs control such that a fluid channel of the fluid channel pipe is cleaned, when it is determined that the fluid channel pipe is contaminated with the foreign materials in the internal region thereof or with the biofilms, and
performs control such that water is introduced into the internal region of the fluid channel pipe, in a state where the fluid channel of the fluid channel pipe is cleaned.

15. The device of claim 14, wherein the processor
controls the switch such that the first light source remains turned on to emit the first light into the internal region in a state where water is introduced into the internal region,
measures the first turbidity value by detecting the scattered light resulting from the first light,
controls the switch such that the second light source remains turned on to emit the second light into the internal region,
measures the second turbidity value by detecting the reflected light resulting from the second light, and
computes the turbidity value for the internal region based on the first turbidity value and the second turbidity value, when the measured first turbidity value or the measured second turbidity value remains normal.

16. A method of sensing water quality, the method being performed by a processor of a device for sensing water quality, the method comprising:
controlling a switch such that a first light source arranged at a first point on a fluid channel pipe remains turned on to emit first light into an internal region of the fluid channel pipe;
measuring a first turbidity value by enabling a detector to detect scattered light resulting from the first light being scattered within the internal region;
controlling the switch such that a second light source arranged at a second point on the fluid channel pipe remains turned on to emit second light into the internal region;
measuring a second turbidity value by enabling the detector to detect reflected light resulting from the second light being reflected by a surface of the fluid channel pipe; and
computing a turbidity value for the internal region based on the first turbidity value and the second turbidity value.

17. The method of claim 16, further comprising:
determining whether the first turbidity value and the second turbidity value remain in a normal state where a linear relationship defined in the form of a linear function is established, or in an abnormal state; and
determining whether the first turbidity or the second turbidity value exceeds a threshold value in the abnormal state,
wherein the turbidity value for the internal region is computed based on the first turbidity value and the second turbidity value when the first turbidity value and the second turbidity value remain in the normal state.

18. The method of claim 17, further comprising:
performing control such that a fluid channel of the fluid channel pipe is cleaned, when the first turbidity value and the second turbidity value are less than the threshold value;
performing control such that water is introduced into the internal region of the fluid channel pipe, in a state where the fluid channel of the fluid channel pipe is cleaned; and
controlling a temperature adjustment device such that the temperature of the internal region of the fluid channel pipe is controlled, when the first turbidity value or the second turbidity value exceeds the threshold value.

19. An apparatus for purifying water, the apparatus comprising:
a water intake port formed such that water is introduced therethrough;
a device for sensing water quality, the device configured to detect the turbidity of the water flowing through a fluid channel pipe from the water intake port; and
a water supply port formed such that water, the turbidity of which is detected, is discharged therethrough,
wherein the device for sensing water quality comprises:
a fluid channel pipe;
an external light blocking structure formed such that the external light blocking structure surrounds the fluid channel pipe and that the fluid channel pipe is inserted into the external light blocking structure, and configured such that an inlet and an outlet of the fluid channel pipe are exposed at a first lateral surface and a second lateral surface, respectively;
a first light source arranged at a first point on the fluid channel pipe and configured to emit first light into an internal region of the fluid channel pipe;
a second light source arranged at a second point on the fluid channel pipe and configured to emit second light into the internal region of the fluid channel pipe;
a detector configured to detect scattered light resulting from the first light being scattered within the internal region of the fluid channel pipe, and to detect reflected light resulting from the second light being reflected by a surface of the fluid channel pipe; and
a processor operatively coupled to the first light source, the second light source, and the detector, and configured to measure the turbidity of a liquid within the fluid channel pipe, and
wherein the processor
measures a first turbidity value by enabling a detector to detect the scattered light resulting from the first light,
measures a second turbidity value by enabling the detector to detect the reflected light resulting from the second light, and
computes a turbidity value for the internal region based on the first turbidity value and the second turbidity value, and detects whether the fluid channel pipe is contaminated.

20. The apparatus of claim 19, wherein the processor
determines whether the first turbidity value and the second turbidity value remain in a normal state where a linear relationship defined in the form of a linear function is established, or in an abnormal state that deviates from the linear relationship,
computes the turbidity value for the internal region based on the first turbidity value and the second turbidity value when the first turbidity value and the second turbidity value remain normal,
performs control such that a fluid channel of the fluid channel pipe is cleansed, when the first turbidity value and the second turbidity value are less than a threshold value,
performs control such that water is introduced into the internal region of the fluid channel pipe in a state where the fluid channel of the fluid channel pipe is cleaned, and
controls a temperature adjustment device such that the temperature of the internal region of the fluid channel pipe is controlled, when the first turbidity value or the second turbidity value exceeds the threshold value in the abnormal state.
